(19) United States
(12) Patent Application Publication
(10) Pub. No.: US 2022/0211556 A1
(43) Pub. Date: Jul. 7, 2022

(54) LOW-RUNOFF AIRLAID NONWOVEN MATERIALS

(71) Applicant: GEORGIA-PACIFIC NONWOVENS LLC, Alanta, GA (US)

(72) Inventor: Jacek K. DUTKIEWICZ, Atlanta, GA (US)

(21) Appl. No.: 17/615,511

(22) PCT Filed: May 28, 2020
(86) PCT No.: PCT/IB2020/055087

§ 371 Date: Nov. 30, 2021

Related U.S. Application Data
(60) Provisional application No. 62/854,546, filed on May 30, 2019.

(51) Int. Cl. A61F 13/537; B32B 5/14; B32B 5/02; B32B 5/26; B32B 7/12; B32B 3/30; B32B 5/08; D04H 1/732

(52) U.S. Cl. CPC A61F 13/537; B32B 2307/72; B32B 5/022; B32B 5/266; B32B 7/12; B32B 3/30; B32B 5/08; D04H 1/732; B32B 2307/732; B32B 2262/04; B32B 2262/124; B32B 2262/144; B32B 2250/02; B32B 2250/03; B32B 2250/20; B32B 2555/02; B32B 2262/02; B32B 5/145

(57) ABSTRACT

Nonwoven materials providing for low runoff and methods of making the same are provided. Such nonwoven materials can be absorbent and include a three-dimensional pattern on one or more surfaces thereof. Such materials can be airlaid and can include multiple layers, comprised of cellulose fibers and synthetic fibers. The nonwoven material can have a percent runoff of less than about 5%.

### CROSS REFERENCE TO RELATED APPLICATION

This application claims the benefit of priority to U.S. Provisional Application No. 62/854,546 filed on May 30, 2019, the contents of which are hereby incorporated by reference in their entirety.

### 1. FIELD

The presently disclosed subject matter relates to nonwoven materials and methods of making the same. Such nonwoven materials can be absorbent and advantageously provide for low runoff. More particularly, such structures include a three-dimensional pattern on one or more surfaces thereof.

### 2. BACKGROUND

Nonwoven structures are important in a wide range of consumer products, such as absorbent articles including baby diapers, adult incontinence products, sanitary napkins, and the like. In certain nonwoven articles, there is often an absorbent core to receive and retain body liquids. The absorbent core is usually disposed between a liquid pervious topsheet, whose function is to allow the passage of fluid to the core and a liquid impervious backsheet whose function is to contain the fluid and to prevent it from passing through the absorbent article to the garment of the wearer of the absorbent article. In certain nonwoven articles, an acquisition-distribution layer (ADL) can be used in combination with the absorbent core.

Such nonwoven structures have runoff properties. Runoff reflects the tendency of a fluid such as menses or urine to run over the surface of the absorbent material before the fluid is fully acquired by the absorbent. This may lead to undesirable leakages from the finished absorbent products such as feminine sanitary napkins, pantiliners, adult incontinence devices, and the like.

There has been a constant demand and remains a need to provide nonwoven materials with low runoff characteristics to reduce or prevent undesirable leakages while exhibiting the desired characteristics of liquid acquisition, distribution and storage. The disclosed subject matter addresses these and other needs.

### 3. SUMMARY OF THE INVENTION

The presently disclosed subject matter provides for improved nonwoven materials which advantageously have low runoff. Such nonwoven materials can include at least one three-dimensional patterned surface.

The present disclosure provides for airlaid nonwoven materials. Such materials can include a first layer and a second layer. The first layer can include bicomponent fibers. The second layer can be disposed adjacent to the first layer and include cellulose fibers and bicomponent fibers. The second layer can be bonded on at least a portion of its outer surface with a binder and at least a portion of the second layer can be patterned. The nonwoven material can have a percent runoff of less than about 5%.

In certain embodiments, the patterning of the second layer can include alternating ridges and valleys, and the ridges can have a higher basis weight than the valleys. The ridges can be from about 2 mm to about 4 mm wide and the valleys can be from about 1 mm to about 2.5 mm wide.

The present disclosure provides for airlaid nonwoven materials. Such materials can include a first layer, a second layer, and a third layer. The first layer can include bicomponent fibers. The second layer can be disposed adjacent to the first layer and can include cellulose fibers and bicomponent fibers. The third layer can be disposed adjacent to the second layer and include cellulose fibers and bicomponent fibers. The third layer can be bonded on at least a portion of its outer surface with a binder. At least a portion of at least one of the first and third layers can be patterned. The nonwoven material can have a percent runoff of less than about 5%

In certain embodiments, the patterning of at least one or the first and third layers can include alternating ridges and valleys, and the ridges can have a higher basis weight than the valleys. The ridges can be from about 2 mm to about 4 mm wide and the valleys can be from about 1 mm to about 2.5 mm wide.

The present disclosure provides for airlaid nonwoven materials. Such materials can include a first layer, a second layer, a third layer, and a layer of super absorbent polymer. The first layer can include bicomponent fibers. The second layer can be disposed adjacent to the first layer and can include cellulose fibers and bicomponent fibers. The third layer can be disposed adjacent to the second layer and can include eucalyptus fibers and bicomponent fibers. The layer of super absorbent polymer can be disposed between the second and third layers. The third layer can be bonded on at least a portion of its outer surface with a binder. At least a portion of the first layer can be patterned. The nonwoven material can have a percent runoff of less than about 5%.

In certain embodiments, the patterning of the first layer can include alternating ridges and valleys, and the ridges can have a higher basis weight than the valleys. The ridges can be from about 2 mm to about 4 mm wide and the valleys can be from about 1 mm to about 2.5 mm wide.

The present disclosure provides for airlaid nonwoven materials. Such materials can include a first layer and a second layer. The first layer can include synthetic fibers. The second layer can be disposed adjacent to the first layer and include cellulose fibers and synthetic fibers. The nonwoven material can be patterned on at least a portion of at least one surface. The nonwoven material can have a percent runoff of less than about 5%. In particular embodiments, the nonwoven material can have a percent runoff of less than about 1%.

In certain embodiments, the nonwoven material can further include a third layer. The third layer can be disposed adjacent to the second layer and include cellulose fibers and synthetic fibers.

In certain embodiments, the nonwoven material can include a layer of superabsorbent polymer. The layer of superabsorbent polymer can be disposed between the second and third layers.

In certain embodiments, the second layer can be bonded on at least a portion of its outer surface with a binder. In alternative embodiments, the third layer can be bonded on at least a portion of its outer surface with a binder.

In certain embodiments, the cellulose fibers of the third layer can include eucalyptus fibers. In certain embodiments, the synthetic fibers of the first and second layers can include bicomponent fibers.

The present disclosure provides for airlaid nonwoven materials. Such materials can include a first layer, a second layer, and a third layer. The first layer can include synthetic fibers. The second layer can be disposed adjacent to the first layer and include cellulose fibers and synthetic fibers. The third layer can be disposed adjacent to the second layer and include cellulose fibers and synthetic fibers. The nonwoven material can be patterned on at least a portion of at least one surface. The nonwoven material can have a percent runoff of less than about 5%. In particular embodiments, the nonwoven material can have a percent runoff of less than about 1%.

In certain embodiments, the nonwoven material can include a layer of superabsorbent polymer. The layer of superabsorbent polymer can be disposed between the second and third layers.

In certain embodiments, the third layer can be bonded on at least a portion of its outer surface with a binder. In certain embodiments, the cellulose fibers of the third layer can include eucalyptus fibers.

The presently disclosure also provides absorbent articles including such nonwoven materials.

The foregoing has outlined broadly the features and technical advantages of the present application in order that the detailed description that follows may be better understood.

Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 4. BRIEF DESCRIPTION OF THE DRAWINGS

FIG. 1A schematically illustrates a composition of a nonwoven material (Structure 2) prepared in accordance with certain non-limiting embodiments as provided in Example 1;

FIG. 1B schematically illustrates a composition of a nonwoven material (Structure 4) prepared in accordance with certain non-limiting embodiments as provided in Example 1;

FIG. 1C schematically illustrates a composition of a nonwoven material (Structure 5) prepared in accordance with certain non-limiting embodiments as provided in Example 1;

FIG. 1D schematically illustrates a composition of a nonwoven material (Structure 7) prepared in accordance with certain non-limiting embodiments as provided in Example 1;

FIG. 2 depicts the runoff testing results of nonwoven materials (Structures 1 and 2) in accordance with Examples 1 and 2;

FIG. 3 depicts the runoff testing results of nonwoven materials (Structures 3-5) in accordance with Examples 1 and 2;

FIG. 4 depicts the runoff testing results of nonwoven materials (Structures 6-7) in accordance with Examples 1 and 2; and

FIG. 5 depicts an exemplary image of a nonwoven material formed with a patterned forming wire (e.g., Ribtech 84, Albany International, Rochester, N.H.).

### 5. DETAILED DESCRIPTION

The presently disclosed subject matter provides novel nonwoven materials having a low runoff and methods of making the same. Nonwoven materials of the present disclosure can include a pattern on at least one surface thereof which surprisingly and advantageously provided nonwoven materials with low runoff. These and other aspects of the disclosed subject matter are discussed more in the detailed description and Examples.

### Definitions

The terms used in this specification generally have their ordinary meanings in the art, within the context of this subject matter and in the specific context where each term is used. Certain terms are defined below to provide additional guidance in describing the compositions and methods of the disclosed subject matter and how to make and use them.

As used in the specification and the appended claims, the singular forms “a,” “an” and “the” include plural referents unless the context clearly dictates otherwise. Thus, for example, reference to “a compound” includes mixtures of compounds.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to systems or processes, the term can mean within an order of magnitude, preferably within 5-fold, and more preferably within 2-fold, of a value.

The term “basis weight” as used herein refers to the quantity by weight of a compound over a given area. Examples of the units of measure include grams per square meter as identified by the acronym “gsm”.

As used herein, the term “cellulose” or “cellulosic” includes any material having cellulose as a major constituent, and specifically, comprising at least 50 percent by weight cellulose or a cellulose derivative. Thus, the term includes cotton, typical wood pulps, cellulose acetate, rayon, thermochemical wood pulp, chemical wood pulp, debonded chemical wood pulp, milkweed floss, microcrystalline cellulose, microfibrillated cellulose, and the like.

As used herein, the phrase “chemically modified,” when used in reference to a fiber, means that the fiber has been treated with a polyvalent metal-containing compound to produce a fiber with a polyvalent metal-containing compound bound to it. It is not necessary that the compound chemically bond with the fibers, although it is preferred that the compound remain associated in close proximity with the fibers, by coating, adhering, precipitation, or any other mechanism such that it is not dislodged from the fibers during normal handling of the fibers. In particular, the compound can remain associated with the fibers even when wetted or washed with a liquid. For convenience, the association between the fiber and the compound may be referred to as the bond, and the compound may be said to be bound to the fiber.

As used herein, the term “fiber” or “fibrous” refers to a particulate material wherein the length to diameter ratio of such particulate material is greater than about 10. Conversely, a “nonfiber” or “nonfibrous” material is meant to refer to a particulate material wherein the length to diameter ratio of such particulate matter is about 10 or less.

As used herein, a “nonwoven” refers to a class of material, including but not limited to textiles or plastics. Nonwovens are sheet or web structures made of fiber, filaments, molten plastic, or plastic films bonded together mechanically, thermally, or chemically. A nonwoven is a fabric made directly from a web of fiber, without the yarn preparation necessary for weaving or knitting. In a nonwoven, the assembly of fibers is held together by one or more of the following: (1) by mechanical interlocking in a random web or mat; (2) by fusing of the fibers, as in the case of thermoplastic fibers; or (3) by bonding with a cementing medium such as a natural or synthetic resin.

As used herein, the term “runoff” refers to the tendency of a fluid to run over the surface of the absorbent material before the fluid is fully acquired by the absorbent. Runoff can be expressed in percent runoff.

As used herein, the term “weight percent” is meant to refer to either (i) the quantity by weight of a constituent/component in the material as a percentage of the weight of a layer of the material; or (ii) to the quantity by weight of a constituent/component in the material as a percentage of the weight of the final nonwoven material or product.

### Fibers

Nonwoven materials of the presently disclosed subject matter comprise fibers. The fibers can be natural, synthetic, or a mixture thereof. In certain embodiments, the fibers can be cellulose-based fibers, one or more synthetic fibers, or a mixture thereof.

### Cellulose Fibers

Any cellulose fibers known in the art, including cellulose fibers of any natural origin, such as those derived from wood pulp or regenerated cellulose, can be used in a cellulosic layer. In certain embodiment, cellulose fibers include, but are not limited to, digested fibers, such as kraft, prehydrolyzed kraft, soda, sulfite, chemi-thermal mechanical, and thermo-mechanical treated fibers, derived from softwood, hardwood or cotton linters. In other embodiments, cellulose fibers include, but are not limited to, kraft digested fibers, including prehydrolyzed kraft digested fibers. Non-limiting examples of cellulose fibers suitable for use in this subject matter are the cellulose fibers derived from softwoods, such as pines, firs, and spruces. Other suitable cellulose fibers include, but are not limited to, those derived from Esparto grass, bagasse, kemp, flax, hemp, kenaf, and other lignaceous and cellulosic fiber sources. Suitable cellulose fibers include, but are not limited to, bleached Kraft southern pine fibers sold under the trademark FOLEY FLUFFS® (Buckeye Technologies Inc., Memphis, Tenn.). Additionally, fibers sold under the trademark CELLU TISSUE® (e.g., Grade 3024) (Clearwater Paper Corporation, Spokane, Wash.) are utilized in certain aspects of the disclosed subject matter.

The nonwoven materials of the disclosed subject matter can also include, but are not limited to, a commercially available bright fluff pulp including, but not limited to, southern softwood kraft (such as Golden Isles® 4725 from GP Cellulose) or southern softwood fluff pulp (such as Treated FOLEY FLUFFS®) northern softwood sulfite pulp (such as T 730 from Weyerhaeuser), or hardwood pulp (such as Eucalyptus). In certain embodiments, the nonwoven materials can include Eucalyptus fibers (Suzano, untreated). While certain pulps may be preferred based on a variety of factors, any absorbent fluff pulp or mixtures thereof can be used. In certain embodiments, wood cellulose, cotton linter pulp, chemically modified cellulose such as crosslinked cellulose fibers and highly purified cellulose fibers can be used. Non-limiting examples of additional pulps are FOLEY FLUFFS® FFTAS (also known as FFTAS or Buckeye Technologies FFT-AS pulp) and Weyco CF401.

In certain embodiments, fine fibers, such as certain softwood fibers can be used. Certain non-limiting examples of such fine fibers, with pulp fiber coarseness properties are provided in Table I below with reference to Watson, P., et al., Canadian Pulp Fibre Morphology: Superiority and Considerations for End Use Potential, The Forestry Chronicle, Vol. 85 No. 3, 401-408 May/June 2009.

TABLE I
Softwood Fibers

| Species | Pulp Fiber Coarseness (mg/100 m) |
|---|---|
| Coastal Douglas-fir | 24 |
| Western hemlock | 20 |
| Spruce/pine | 18 |
| Western redcedar | 16 |
| Southern pine | 30 |
| Radiata pine | 22 |
| Scandinavian pine | 20 |
| Black spruce | 18 |

In certain embodiments, fine fibers, such as certain hardwood fibers can be used. Certain non-limiting examples of such fine fibers, with pulp fiber coarseness properties are provided in Table II with reference, at least in part, to Horn, R., Morphology of Pulp Fiber from Hardwoods and Influence on Paper Strength, Research Paper FPL 312, Forest Products Laboratory, U.S. Department of Agriculture (1978) and Bleached Eucalyptus Kraft Pulp ECF Technical Sheet (April 2017) (available at: https://www.metsafibre.com/en/Documents/Data-sheets/Cenibra-euca-Eucalyptus.pdf). In particular embodiments, Eucalyptus pulp (Sunzano, untreated) can be used.

TABLE II
Hardwood Fibers

| Species | Pulp Fiber Coarseness (mg/100 m) |
|---|---|
| Red alder | 12.38 |
| Aspen | 8.59 |
| American elm | 9.53 |
| Paper birch | 13.08 |
| American beech | 13.10 |
| Shagbark hickory | 10.59 |
| Sugar maple | 7.86 |
| White oak | 14.08 |
| Eucalyptus | 6.5 +/− 2.3 |

Other suitable types of cellulose fiber include, but are not limited to, chemically modified cellulose fibers. In particular embodiments, the modified cellulose fibers are crosslinked cellulose fibers. U.S. Pat. Nos. 5,492,759, 5,601,921, and 6,159,335, all of which are hereby incorporated by reference in their entireties, relate to chemically treated cellulose fibers useful in the practice of this disclosed subject matter. In certain embodiments, the modified cellulose fibers comprise a polyhydroxy compound. Non-limiting examples of polyhydroxy compounds include glycerol, trimethylolpropane, pentaerythritol, polyvinyl alcohol, partially hydrolyzed polyvinyl acetate, and fully hydrolyzed polyvinyl acetate. In certain embodiments, the fiber is treated with a polyvalent cation-containing compound. In one embodiment, the polyvalent cation-containing compound is present in an amount from about 0.1 weight percent to about 20 weight percent based on the dry weight of the untreated fiber. In particular embodiments, the polyvalent cation containing compound is a polyvalent metal ion salt. In certain embodiments, the polyvalent cation containing compound is selected from the group consisting of aluminum, iron, tin, salts thereof, and mixtures thereof. Any polyvalent metal salt including transition metal salts may be used. Non-limiting examples of suitable polyvalent metals include beryllium, magnesium, calcium, strontium, barium, titanium, zirconium, vanadium, chromium, molybdenum, tungsten, manganese, iron, cobalt, nickel, copper, zinc, aluminum and tin. Preferred ions include aluminum, iron and tin. The preferred metal ions have oxidation states of +3 or +4. Any salt containing the polyvalent metal ion may be employed. Non-limiting examples of suitable inorganic salts of the above metals include chlorides, nitrates, sulfates, borates, bromides, iodides, fluorides, nitrides, perchlorates, phosphates, hydroxides, sulfides, carbonates, bicarbonates, oxides, alkoxides phenoxides, phosphites, and hypophosphites. Non-limiting examples of suitable organic salts of the above metals include formates, acetates, butyrates, hexanoates, adipates, citrates, lactates, oxalates, propionates, salicylates, glycinates, tartrates, glycolates, sulfonates, phosphonates, glutamates, octanoates, benzoates, gluconates, maleates, succinates, and 4,5-dihydroxy-benzene-1,3-disulfonates. In addition to the polyvalent metal salts, other compounds such as complexes of the above salts include, but are not limited to, amines, ethylenediaminetetra-acetic acid (EDTA), diethylenetriaminepenta-acetic acid (DIPA), nitrilotri-acetic acid (NTA), 2,4-pentanedione, and ammonia may be used.

In one embodiment, the cellulose pulp fibers are chemically modified cellulose pulp fibers that have been softened or plasticized to be inherently more compressible than unmodified pulp fibers. The same pressure applied to a plasticized pulp web will result in higher density than when applied to an unmodified pulp web. Additionally, the densified web of plasticized cellulose fibers is inherently softer than a similar density web of unmodified fiber of the same wood type. Softwood pulps may be made more compressible using cationic surfactants as debonders to disrupt interfiber associations. Use of one or more debonders facilitates the disintegration of the pulp sheet into fluff in the airlaid process. Examples of debonders include, but are not limited to, those disclosed in U.S. Pat. Nos. 4,432,833, 4,425,186, and 5,776,308, all of which are hereby incorporated by reference in their entireties. One example of a debonder-treated cellulose pulp is FFLE+. Plasticizers for cellulose, which can be added to a pulp slurry prior to forming wetlaid sheets, can also be used to soften pulp, although they act by a different mechanism than debonding agents. Plasticizing agents act within the fiber, at the cellulose molecule, to make flexible or soften amorphous regions. The resulting fibers are characterized as limp. Since the plasticized fibers lack stiffness, the comminuted pulp is easier to densify compared to fibers not treated with plasticizers. Plasticizers include, but are not limited to, polyhydric alcohols such as glycerol, low molecular weight polyglycol such as polyethylene glycols, and polyhydroxy compounds. These and other plasticizers are described and exemplified in U.S. Pat. Nos. 4,098,996, 5,547,541, and 4,731,269, all of which are hereby incorporated by reference in their entireties. For example and not limitation, the plasticizer can be polyethylene glycol 100 (PEG 100, polyethylene glycol 200 (PEG 200), polyethylene glycol 300 (PEG 300), or polyethylene glycol 400 (PEG 400). Ammonia, urea, and alkylamines are also known to plasticize wood products, which mainly contain cellulose (A. J. Stamm, Forest Products Journal 5(6):413, 1955, hereby incorporated by reference in its entirety).

In particular embodiments of the disclosed subject matter, the following cellulose is used: GP 4723, fully treated pulp from Leaf River, Eucalyptus pulp (Suzano, untreated), or combinations thereof.

Nonwoven materials of the present disclosure can include cellulose fibers. In certain embodiments, one or more layers of the nonwoven material can contain from about 5 gsm to about 150 gsm, about 5 gsm to about 100 gsm, or about 10 gsm to about 50 gsm cellulose fibers. In particular embodiments, one or more layers can contain about 20 gsm, about 21 gsm, about 21.36 gsm, about 30 gsm, about 40 gsm, about 50 gsm, about 60 gsm, about 62 gsm, about 70 gsm, or about 71 gsm cellulose fibers.

### Synthetic Fibers

In addition to the use of cellulose fibers, the presently disclosed subject matter also contemplates the use of synthetic fibers. In one embodiment, the synthetic fibers comprise bicomponent and/or mono-component fibers. Bicomponent fibers having a core and sheath are known in the art. Many varieties are used in the manufacture of nonwoven materials, particularly those produced for use in airlaid techniques. Various bicomponent fibers suitable for use in the presently disclosed subject matter are disclosed in U.S. Pat. Nos. 5,372,885 and 5,456,982, both of which are hereby incorporated by reference in their entireties. Examples of bicomponent fiber manufacturers include, but are not limited to, Trevira (Bobingen, Germany), Fiber Innovation Technologies (Johnson City, Tenn.) and ES Fiber Visions (Athens, Ga.).

Bicomponent fibers can incorporate a variety of polymers as their core and sheath components. Bicomponent fibers that have a PE (polyethylene) or modified PE sheath typically have a PET (polyethylene terephthalate) or PP (polypropylene) core. In one embodiment, the bicomponent fiber has a core made of polyester and sheath made of polyethylene. In another embodiment, the bicomponent fiber has a core made of polypropylene and a sheath made of polyethylene.

The denier of the bicomponent fiber preferably ranges from about 1.0 dpf to about 4.0 dpf, and more preferably from about 1.5 dpf to about 2.5 dpf. The length of the bicomponent fiber can be from about 3 mm to about 36 mm, preferably from about 3 mm to about 12 mm, more preferably from about 3 mm to about 10. In particular embodiments, the length of the bicomponent fiber is from about 4 mm to about 8 mm, or about 6 mm. In a particular embodiment, the bicomponent fiber is Trevira T255 which contains a polyester core and a polyethylene sheath modified with maleic anhydride. T255 has been produced in a variety of deniers, cut lengths and core sheath configurations with preferred configurations having a denier from about 1.7 dpf to 2.0 dpf and a cut length of about 4 mm to 12 mm and a concentric core sheath configuration. In a specific embodiment, the bicomponent fiber is Trevira 1661, T255, 2.0 dpf and 6 mm in length.

Bicomponent fibers are typically fabricated commercially by melt spinning. In this procedure, each molten polymer is extruded through a die, for example, a spinneret, with subsequent pulling of the molten polymer to move it away from the face of the spinneret. This is followed by solidification of the polymer by heat transfer to a surrounding fluid medium, for example chilled air, and taking up of the now solid filament. Non-limiting examples of additional steps after melt spinning can also include hot or cold drawing, heat treating, crimping and cutting. This overall manufacturing process is generally carried out as a discontinuous two-step process that first involves spinning of the filaments and their collection into a tow that comprises numerous filaments. During the spinning step, when molten polymer is pulled away from the face of the spinneret, some drawing of the filament does occur which can also be called the draw-down. This is followed by a second step where the spun fibers are drawn or stretched to increase molecular alignment and crystallinity and to give enhanced strength and other physical properties to the individual filaments. Subsequent steps can include, but are not limited to, heat setting, crimping and cutting of the filament into fibers. The drawing or stretching step can involve drawing the core of the bicomponent fiber, the sheath of the bicomponent fiber or both the core and the sheath of the bicomponent fiber depending on the materials from which the core and sheath are comprised as well as the conditions employed during the drawing or stretching process.

Bicomponent fibers can also be formed in a continuous process where the spinning and drawing are done in a continuous process. During the fiber manufacturing process it is desirable to add various materials to the fiber after the melt spinning step at various subsequent steps in the process. These materials can be referred to as “finish” and be comprised of active agents such as, but not limited to, lubricants and anti-static agents. The finish is typically delivered via an aqueous based solution or emulsion. Finishes can provide desirable properties for both the manufacturing of the bicomponent fiber and for the user of the fiber, for example in an airlaid or wetlaid process.

Numerous other processes are involved before, during and after the spinning and drawing steps and are disclosed in U.S. Pat. Nos. 4,950,541, 5,082,899, 5,126,199, 5,372,885, 5,456,982, 5,705,565, 2,861,319, 2,931,091, 2,989,798, 3,038,235, 3,081,490, 3,117,362, 3,121,254, 3,188,689, 3,237,245, 3,249,669, 3,457,342, 3,466,703, 3,469,279, 3,500,498, 3,585,685, 3,163,170, 3,692,423, 3,716,317, 3,778,208, 3,787,162, 3,814,561, 3,963,406, 3,992,499, 4,052,146, 4,251,200, 4,350,006, 4,370,114, 4,406,850, 4,445,833, 4,717,325, 4,743,189, 5,162,074, 5,256,050, 5,505,889, 5,582,913, and 6,670,035, all of which are hereby incorporated by reference in their entireties.

The presently disclosed subject matter can also include, but are not limited to, articles that contain bicomponent fibers that are partially drawn with varying degrees of draw or stretch, highly drawn bicomponent fibers and mixtures thereof. These can include, but are not limited to, a highly drawn polyester core bicomponent fiber with a variety of sheath materials, specifically including a polyethylene sheath such as Trevira T255 (Bobingen, Germany) or a highly drawn polypropylene core bicomponent fiber with a variety of sheath materials, specifically including a polyethylene sheath such as ES FiberVisions AL-Adhesion-C (Varde, Denmark). Additionally, Trevira T265 bicomponent fiber (Bobingen, Germany), having a partially drawn core with a core made of polybutylene terephthalate (PBT) and a sheath made of polyethylene can be used. The use of both partially drawn and highly drawn bicomponent fibers in the same structure can be leveraged to meet specific physical and performance properties based on how they are incorporated into the structure.

The bicomponent fibers of the presently disclosed subject matter are not limited in scope to any specific polymers for either the core or the sheath as any partially drawn core bicomponent fiber can provide enhanced performance regarding elongation and strength. The degree to which the partially drawn bicomponent fibers are drawn is not limited in scope as different degrees of drawing will yield different enhancements in performance. The scope of the partially drawn bicomponent fibers encompasses fibers with various core sheath configurations including, but not limited to concentric, eccentric, side by side, islands in a sea, pie segments and other variations. The relative weight percentages of the core and sheath components of the total fiber can be varied. In addition, the scope of this subject matter covers the use of partially drawn homopolymers such as polyester, polypropylene, nylon, and other melt spinnable polymers. The scope of this subject matter also covers multicomponent fibers that can have more than two polymers as part of the fiber structure.

In particular embodiments, the bicomponent fibers are low dtex staple bicomponent fibers in the range of about 0.5 dtex to about 20 dtex. In certain embodiments, the dtex value can range from about 1.3 dtex to about 15 dtex, about 1.5 dtex to about 10 dtex, about 1.7 dtex to about 6.7 dtex, or about 2.2 dtex to about 5.7 dtex. In certain embodiments, the dtex value can be about 1.3 dtex, about 1.5 dtex, about 1.7 dtex, about 2.2 dtex, about 3.3 dtex, about 5.7 dtex, about 6.7 dtex, or about 10 dtex. In certain embodiments, the bicomponent fibers are staple fibers forming a web.

Other synthetic fibers suitable for use in various embodiments as fibers or as bicomponent binder fibers include, but are not limited to, fibers made from various polymers including, by way of example and not by limitation, acrylic, polyamides (including, but not limited to, Nylon 6, Nylon 6/6, Nylon 12, polyaspartic acid, polyglutamic acid), polyamines, polyimides, polyacrylics (including, but not limited to, polyacrylamide, polyacrylonitrile, esters of methacrylic acid and acrylic acid), polycarbonates (including, but not limited to, polybisphenol A carbonate, polypropylene carbonate), polydienes (including, but not limited to, polybutadiene, polyisoprene, polynorbomene), polyepoxides, polyesters (including, but not limited to, polyethylene terephthalate, polybutylene terephthalate, polytrimethylene terephthalate, polycaprolactone, polyglycolide, polylactide, polyhydroxybutyrate, polyhydroxyvalerate, polyethylene adipate, polybutylene adipate, polypropylene succinate), polyethers (including, but not limited to, polyethylene glycol (polyethylene oxide), polybutylene glycol, polypropylene oxide, polyoxymethylene (paraformaldehyde), polytetramethylene ether (polytetrahydrofuran), polyepichlorohydrin), polyfluorocarbons, formaldehyde polymers (including, but not limited to, urea-formaldehyde, melamine-formaldehyde, phenol formaldehyde), natural polymers (including, but not limited to, cellulosics, chitosans, lignins, waxes), polyolefins (including, but not limited to, polyethylene, polypropylene, polybutylene, polybutene, polyoctene), polyphenylenes (including, but not limited to, polyphenylene oxide, polyphenylene sulfide, polyphenylene ether sulfone), silicon containing polymers (including, but not limited to, polydimethyl siloxane, polycarbomethyl silane), polyurethanes, polyvinyls (including, but not limited to, polyvinyl butyral, polyvinyl alcohol, esters and ethers of polyvinyl alcohol, polyvinyl acetate, polystyrene, polymethylstyrene, polyvinyl chloride, polyvinyl pryrrolidone, polymethyl vinyl ether, polyethyl vinyl ether, polyvinyl methyl ketone), polyacetals, polyarylates, and copolymers (including, but not limited to, polyethylene-co-vinyl acetate, polyethylene-co-acrylic acid, polybutylene terephthalate-co-polyethylene terephthalate, polylauryllactam-block-polytetrahydrofuran), polybutylene succinate and polylactic acid based polymers.

In specific embodiments, the synthetic fiber layer contains a high dtex staple fibers in the range of about 2 to about 20 dtex. In certain embodiments, the dtex value can range from about 2 dtex to about 15 dtex, or from about 2 dtex to about 10 dtex. In particular embodiments, the fiber can have a dtex value of about 6.7 dtex.

In other specific embodiments, the synthetic layer contains synthetic filaments. The synthetic filaments can be formed by spinning and/or extrusion processes. For example, such processes can be similar to the methods described above with reference to melt spinning processes. The synthetic filaments can include one or more continuous strands. In certain embodiments, the synthetic filaments can include polypropylene.

In particular embodiments of the disclosed subject matter, the following synthetic fiber is used: Trevira Type 255, 6.7 dtex, 6 mm, PE/PET; Trevira Type 245, 6.7 dtex, 3 mm; Trevira PE/PET 70% core, 1.7 dtex, 6 mm; Trevira PE/PET 30% core, 1.5 dtex, 6 mm; or combinations thereof.

Nonwoven materials of the present disclosure can include synthetic fibers. In certain embodiments, one or more layers of the nonwoven material can contain from about 1 gsm to about 40 gsm, about 5 gsm to about 30 gsm, or about 10 gsm to about 25 gsm synthetic fibers. In particular embodiments, one or more layers of the nonwoven material can contain about 6 gsm, about 6.28 gsm, about 8 gsm, about 10 gsm, about 25 gsm, about 26 gsm, about 26.34 gsm, or about 27 gsm synthetic fibers.

### Binders

In certain embodiments, the nonwoven materials described herein can include binders. Suitable binders include, but are not limited to, liquid binders and powder binders. Non-limiting examples of liquid binders include emulsions, solutions, or suspensions of binders. Non-limiting examples of binders include polyethylene powders, copolymer binders, vinylacetate ethylene binders, styrene-butadiene binders, urethanes, urethane-based binders, acrylic binders, thermoplastic binders, natural polymer based binders, and mixtures thereof.

Suitable binders include, but are not limited to, copolymers, vinylacetate ethylene (“VAE”) copolymers, which can have a stabilizer such as Wacker Vinnapas 192, Wacker Vinnapas EF 539, Wacker Vinnapas EP907, Wacker Vinnapas EP129, Celanese Duroset E130, Celanese Dur-O-Set Elite 130 25-1813 and Celanese Dur-O-Set TX-849, Celanese 75-524A, polyvinyl alcohol-polyvinyl acetate blends such as Wacker Vinac 911, vinyl acetate homopolyers, polyvinyl amines such as BASF Luredur, acrylics, cationic acrylamides, polyacryliamides such as Bercon Berstrength 5040 and Bercon Berstrength 5150, hydroxyethyl cellulose, starch such as National Starch CATO RTM 232, National Starch CATO RTM 255, National Starch Optibond, National Starch Optipro, or National Starch OptiPLUS, guar gum, styrene-butadienes, urethanes, urethane-based binders, thermoplastic binders, acrylic binders, and carboxymethyl cellulose such as Hercules Aqualon CMC. In certain embodiments, the binder is a natural polymer based binder. Non-limiting examples of natural polymer based binders include polymers derived from starch, cellulose, chitin, and other polysaccharides.

In certain embodiments, the binder is water-soluble. In one embodiment, the binder is a vinylacetate ethylene copolymer. One non-limiting example of such copolymers is EP907 (Wacker Chemicals, Munich, Germany). Vinnapas EP907 can be applied at a level of about 10% solids incorporating about 0.75% by weight Aerosol OT (Cytec Industries, West Paterson, N.J.), which is an anionic surfactant. Other classes of liquid binders such as styrene-butadiene and acrylic binders can also be used. In certain embodiments, Vinnapas 192 can be applied at a level of about 15% incorporating about 0.08% by weight Aerosol OT 75 (Cytec Industries, West Paterson, N.J.).

In certain embodiments, the binder is not water-soluble. Examples of these binders include, but are not limited to, Vinnapas 124 and 192 (Wacker), which can have an opacifier and whitener, including, but not limited to, titanium dioxide, dispersed in the emulsion. Other binders include, but are not limited to, Celanese Emulsions (Bridgewater, N.J.) Elite 22 and Elite 33.

In certain embodiments, the binder is a thermoplastic binder. Such thermoplastic binders include, but are not limited to, any thermoplastic polymer which can be melted at temperatures which will not extensively damage the cellulose fibers. Preferably, the melting point of the thermoplastic binding material will be less than about 175° C. Examples of suitable thermoplastic materials include, but are not limited to, suspensions of thermoplastic binders and thermoplastic powders. In particular embodiments, the thermoplastic binding material can be, for example, polyethylene, polypropylene, polyvinylchloride, and/or polyvinylidene chloride.

In particular embodiments, the vinylacetate ethylene binder is non-crosslinkable. In one embodiment, the vinylacetate ethylene binder is crosslinkable. In certain embodiments, the binder is WD4047 urethane-based binder solution supplied by HB Fuller. In one embodiment, the binder is Michem Prime 4983-45N dispersion of ethylene acrylic acid (“EAA”) copolymer supplied by Michelman. In certain embodiments, the binder is Dur-O-Set Elite 22LV emulsion of VAE binder supplied by Celanese Emulsions (Bridgewater, N.J.). As noted above, in particular embodiments, the binder is crosslinkable. It is also understood that crosslinkable binders are also known as permanent wet strength binders. A permanent wet-strength binder includes, but is not limited to, Kymene® (Hercules Inc., Wilmington, Del.), Parez® (American Cyanamid Company, Wayne, N.J.), Wacker Vinnapas or AF192 (Wacker Chemie AG, Munich, Germany), or the like. Various permanent wet-strength agents are described in U.S. Pat. Nos. 2,345,543, 2,926,116, and 2,926,154, the disclosures of which are incorporated by reference in their entirety. Other permanent wet-strength binders include, but are not limited to, polyamine-epichlorohydrin, polyamide epichlorohydrin or polyamide-amine epichlorohydrin resins, which are collectively termed “PAE resins”. Non-limiting exemplary permanent wet-strength binders include Kymene 557H or Kymene 557LX (Hercules Inc., Wilmington, Del.) and have been described in U.S. Pat. Nos. 3,700,623 and 3,772,076, which are incorporated herein in their entirety by reference thereto.

Alternatively, in certain embodiments, the binder is a temporary wet-strength binder. The temporary wet-strength binders include, but are not limited to, Hercobond® (Hercules Inc., Wilmington, Del.), Parez® 750 (American Cyanamid Company, Wayne, N.J.), Parez® 745 (American Cyanamid Company, Wayne, N.J.), or the like. Other suitable temporary wet-strength binders include, but are not limited to, dialdehyde starch, polyethylene imine, mannogalactan gum, glyoxal, and dialdehyde mannogalactan. Other suitable temporary wet-strength agents are described in U.S. Pat. Nos. 3,556,932, 5,466,337, 3,556,933, 4,605,702, 4,603,176, 5,935,383, and 6,017,417, all of which are incorporated herein in their entirety by reference thereto.

In particular embodiments of the disclosed subject matter, the following binder is used: Vinnapas 192, Wacker with 0.20 gsm of surfactant Aerosol OT 75, Cytec Industries or Vinnapas 192, Wacker with 0.8% surfactant Aerosol OT 75, Cytec Industries.

In certain embodiments, binders can be applied as emulsions in amounts ranging from about 1 gsm to about 10 gsm, about 1 gsm to about 8 gsm, about 1 gsm to about 5 gsm, about 1 gsm to about 4 gsm, about 5 gsm to about 10 gsm, about 2 gsm to about 5 gsm, or about 2 gsm to about 3 gsm. In particular embodiments, binders can be applied as emulsions in an amount of about 1 gsm, about 2 gsm, about 3 gsm, about 4 gsm, about 5 gsm, about 6 gsm, or about 6.02 gsm. Binders can be applied to one side of a fibrous layer, preferably an externally facing layer. Alternatively, binder can be applied to both sides of a layer, in equal or disproportionate amounts. In certain embodiments, binders can be applied to at least one outer surface of a nonwoven material. In particular embodiments, binders can be applied at least two outer surfaces of a nonwoven material.

### Other Additives

The materials of the presently disclosed subject matter can also contain other additives. For example, the materials can contain superabsorbent polymer (SAP). The types of superabsorbent polymers which may be used in the disclosed subject matter include, but are not limited to, SAPs in their particulate form such as powder, irregular granules, spherical particles, staple fibers and other elongated particles. U.S. Pat. Nos. 5,147,343, 5,378,528, 5,795,439, 5,807,916, 5,849,211, and 6,403,857, which are hereby incorporated by reference in their entireties, describe various superabsorbent polymers and methods of making superabsorbent polymers. One example of a superabsorbent polymer forming system is crosslinked acrylic copolymers of metal salts of acrylic acid and acrylamide or other monomers such as 2-acrylamido-2-methylpropanesulfonic acid. Many conventional granular superabsorbent polymers are based on poly(acrylic acid) which has been crosslinked during polymerization with any of a number of multi-functional co-monomer crosslinking agents well-known in the art. Examples of multi-functional crosslinking agents are set forth in U.S. Pat. Nos. 2,929,154, 3,224,986, 3,332,909, and 4,076,673, which are incorporated herein by reference in their entireties. For instance, crosslinked carboxylated polyelectrolytes can be used to form superabsorbent polymers. Other water-soluble polyelectrolyte polymers are known to be useful for the preparation of superabsorbents by crosslinking, these polymers include: carboxymethyl starch, carboxymethyl cellulose, chitosan salts, gelatine salts, etc. They are not, however, commonly used on a commercial scale to enhance absorbency of dispensable absorbent articles mainly due to their higher cost. Superabsorbent polymer granules useful in the practice of this subject matter are commercially available from a number of manufacturers, such as BASF, Dow Chemical (Midland, Mich.), Stockhausen (Greensboro, N.C.), Chemdal (Arlington Heights, Ill.), and Evonik (Essen, Germany). Non-limiting examples of SAP include a surface crosslinked acrylic acid based powder such as Stockhausen 9350 or SX70, BASF Hysorb Fem 33, BASF HySorb FEM 33N, or Evonik Favor SXM 7900.

In particular embodiments of the disclosed subject matter, the following additive is used: Evonik Favor SXM 7900.

In certain embodiments, the other additives can be used in a layer in amounts ranging from about 5% to about 50% based on the total weight of the structure. In certain embodiments, the content of the other additives is between about 0% and about 30%, about 0% and about 15%, about 5% and about 25%, about 5% and about 15%, or about 10% and about 20%, based on a total weight of the structure. In particular embodiments, the content of other additives can be about 0%, about 2%, about 5%, about 8%, about 10%, about 15%, about 20%, about 25%, or about 30%, based on a total weight of the structure. In certain embodiments, the amount of additives in a layer can range from about 5 gsm to about 50 gsm, about 5 gsm to about 25 gsm, about 10 gsm to about 50 gsm, or about 12 gsm to about 40 gsm, or about 15 gsm to about 25 gsm. In particular embodiments, the amount of other additives can be used in a layer in an amount of about 10 gsm. For example, in certain embodiments, the nonwoven material can include SAP in an amount of about 10 gsm.

### Nonwoven Materials

The presently disclosed subject matter provides for nonwoven materials having reduced runoff. Such nonwoven materials can include a three-dimensional pattern on at least one surface thereof. As embodied herein, the nonwoven material can include at least one layer, at least two layers, or at least three layers. In certain embodiments, the nonwoven material can include more than three layers. In particular embodiments, the nonwoven material includes two layers or three layers. As further embodied herein, the nonwoven material can be an airlaid material. In particular embodiments, the nonwoven material can be an absorbent structure for liquid acquisition and temporary storage; for liquid acquisition, distribution and permanent storage; or for liquid distribution and permanent storage.

Nonwoven materials of the present disclosure can be an absorbent structure for liquid acquisition and temporary storage. In certain embodiments, the nonwoven material can have at least one layer. The at least one layer can include cellulose fibers, synthetic fibers, or combinations thereof. In certain embodiments, the nonwoven material can have at least two layers. In such embodiments, the nonwoven material can include at least one layer including synthetic fibers, for example, bonded synthetic fibers such as bicomponent fibers, monocomponent fibers, staple fibers, staple fibers forming a fibrous web prefabricated in a carding process, or the like. The nonwoven material can further include an additional layer including synthetic fibers, for example, bonded synthetic fibers such as eccentric bicomponent fibers, monocomponent fibers, staple fibers forming a fibrous web prefabricated in a carding process, or the like. In particular embodiments, at least one layer can further include cellulosic fibers such as softwood or hardwood fibers. In certain embodiments at least one layer can include synthetic fibers such as bicomponent or monocomponent synthetic fibers and be bonded with a binder. The binder can be applied, for example, by spraying and drying a binder emulsion. The nonwoven material can further include a three-dimensional pattern on at least one surface of the nonwoven material. In certain embodiments, the nonwoven material can include a three-dimensional pattern on a bottom or lower surface. In alternative embodiments, the nonwoven material can include a three-dimensional pattern on an upper or top surface.

Nonwoven materials of the present disclosure can be an absorbent structure for liquid acquisition, distribution and permanent storage. In certain embodiments, the nonwoven material can have at least one layer. The at least one layer can include cellulose fibers, synthetic fibers, or combinations thereof. In certain embodiments, the nonwoven material can have at least two layers. In such embodiments, the nonwoven material can include at least one layer comprising bonded synthetic fibers such as eccentric bicomponent fibers, monocomponent fibers, staple fibers forming a fibrous web prefabricated in a carding process, or the like. In certain embodiments, the nonwoven material can further include at least one layer including cellulosic fibers such as softwood or hardwood fibers. In particular embodiments, at least one layer of the nonwoven material includes wood fibers having coarseness lower than 15 mg/100 m such as Eucalyptus fibers. In such embodiments, the layer including the cellulosic fibers can be a lower or bottom layer of the nonwoven material. In particular embodiments, the nonwoven material can include a lower or bottom layer including cellulosic fibers such as softwood or hardwood fibers. In particular embodiments, the nonwoven material can include a lower or bottom layer including wood fibers having coarseness lower than about 15 mg/100 m such as Eucalyptus fibers. The at least one layer comprising cellulosic fibers can further include synthetic fibers and can be bonded with a binder. The binder can be applied, for example, by spraying and drying a binder emulsion. In certain embodiments, the nonwoven material can include a three-dimensional pattern on a bottom or lower surface. In alternative embodiments, the nonwoven material can include a three-dimensional pattern on an upper or top surface. Such nonwoven materials can further include an intermediate layer disposed between layers. In certain embodiments, the intermediate layer can include cellulosic fibers. In particular embodiments, the intermediate layer can include cellulosic fibers bonded with bicomponent fibers. Such intermediate layer can further include one or more additives. In certain embodiments, the one or more additives can include super absorbent particles (SAP) in the form of granules or fibers. The super absorbent particles (SAP) can be blended with the cellulosic and/or synthetic fibers or they can form one or more layers disposed between other layers of the nonwoven structure.

Nonwoven materials of the present disclosure can be an absorbent structure for liquid distribution and permanent storage. In certain embodiments, the nonwoven material can have at least one layer. The nonwoven material can include at least one layer including cellulosic fibers bonded with bicomponent fibers. At least one surface of the nonwoven structure can be bonded with a binder. In particular embodiments, at least two surfaces of the nonwoven structure can be bonded with a binder. The binder can be applied, for example, by spraying and drying a binder emulsion. In certain embodiments, the nonwoven material can further include one or more additives. In certain embodiments, the one or more additives can include super absorbent particles (SAP) in the form of granules or fibers. The super absorbent particles (SAP) can be blended with the cellulosic and/or synthetic fibers or they can form one or more layers disposed between other layers of the nonwoven structure. In certain embodiments, the nonwoven material can include a three-dimensional pattern on a bottom or lower surface. In alternative embodiments, the nonwoven material can include a three-dimensional pattern on an upper or top surface.

In certain embodiments, the nonwoven material can be coated on at least of a portion of its outer surface with a binder. It is not necessary that the binder chemically bond with a portion of the layer, although it is preferred that the binder remain associated in close proximity with the layer, by coating, adhering, precipitation, or any other mechanism such that it is not dislodged from the layer during normal handling of the layer. For convenience, the association between the layer and the binder discussed above can be referred to as the bond, and the compound can be said to be bonded to the layer. If present, the binder can be applied in amounts ranging from about 1 gsm to about 15 gsm, or from about 2 gsm to about 10 gsm, or from about 2 gsm to about 8 gsm, or from about 3 gsm to about 5 gsm. Binders can be applied to one side of a fibrous layer, preferably an externally facing layer. In certain embodiments, binders can be applied to at least one outer surface of a nonwoven material.

In particular embodiments, the nonwoven material comprises at least two layers, wherein each layer comprises a specific fibrous content. In specific embodiments, the nonwoven material can be a two-layered nonwoven structure. The nonwoven material can include a synthetic fiber layer and a blended layer comprising cellulosic fibers and synthetic fibers. The first layer can include synthetic fibers such as bicomponent fibers. In particular embodiments, the first layer can include eccentric bicomponent fibers. The second layer can be disposed adjacent to the first layer. The second layer can include a blend of cellulosic fibers and synthetic fibers. In particular embodiments, the second layer can include a blend of cellulosic fibers and bicomponent fibers. The second layer can be bonded on at least a portion of its outer surface with a binder.

In particular embodiments, the nonwoven material comprises at least three layers, wherein each layer comprises a specific fibrous content. In specific embodiments, the nonwoven material can be a three-layered nonwoven structure. The nonwoven material can include a synthetic fiber layer and at least one layer including a blend of cellulosic fibers and bicomponent fibers. In certain embodiments, at least one layer includes cellulosic fibers comprising Eucalyptus fibers. The first layer can include synthetic fibers such as bicomponent fibers. In particular embodiments, the first layer can include eccentric bicomponent fibers. The second and third layers can include a blend of cellulosic fibers and synthetic fibers. In particular embodiments, the second and third layers can include a blend of cellulosic fibers and bicomponent fibers. In certain embodiments, the cellulosic fibers of the third layer can include wood fibers having coarseness lower than about 15 mg/100 m such as Eucalyptus fibers. The third layer can be bonded on at least a portion of its outer surface with a binder.

In particular embodiments, the nonwoven material comprises at least three layers, wherein each layer comprises a specific fibrous content. In specific embodiments, the nonwoven material can be a three-layered nonwoven structure. The nonwoven material can include a synthetic fiber layer and at least one layer including a blend of cellulosic fibers and bicomponent fibers. In certain embodiments, the nonwoven materials can further include one or more additives such as super absorbent polymer (SAP). The first layer can include synthetic fibers such as bicomponent fibers. In particular embodiments, the first layer can include eccentric bicomponent fibers. The second and third layers can include a blend of cellulosic fibers and synthetic fibers. In particular embodiments, the second and third layers can include a blend of cellulosic fibers and bicomponent fibers. In certain embodiments, the cellulosic fibers of the third layer can include wood fibers having coarseness lower than about 15 mg/100 m such as Eucalyptus fibers. The third layer can be bonded on at least a portion of its outer surface with a binder. In certain embodiments, the nonwoven material can further include a layer of one or more additives disposed between the second and third layers. In particular embodiments, the one or more additives can include super absorbent polymer (SAP).

Nonwoven materials of the present disclosure can include at least two layers or at least three layers, wherein each layer comprises a specific fibrous content. In certain embodiments, the first layer can include synthetic fibers in an amount of about 5 gsm to about 60 gsm, about 10 gsm to about 50 gsm, or about 15 gsm to about 30 gsm. In particular embodiments, the first layer can include synthetic fibers in an amount of about 20 gsm, about 25 gsm, about 26 gsm, or about 26.34 gsm. In certain embodiments, the second layer can include a blend of cellulosic fibers and bicomponent fibers. The cellulosic fibers can be present in the second layer in an amount of about 10 gsm to about 90 gsm, about 15 gsm to about 80 gsm, or about 50 gsm to about 75 gsm. In particular embodiments, the first layer can include about 20 gsm, about 21 gsm, about 25 gm, about 50 gsm, about 62 gsm, or about 71 gsm cellulose fibers. The synthetic fibers can be present in the second layer in an amount of about 1 gsm to about 50 gsm, about 5 gsm to about 35 gsm, or about 5 gsm to about 30 gsm. In particular embodiments, the first layer can include about 5 gsm, about 6 gsm, about 15 gsm, about 20 gsm, or about 27 gsm synthetic fibers. In certain embodiments, the third layer can include a blend of cellulosic fibers and bicomponent fibers. The cellulosic fibers can be present in the third layer in an amount of about 10 gsm to about 90 gsm, about 15 gsm to about 80 gsm, or about 50 gsm to about 75 gsm. In particular embodiments, the first layer can include about 20 gsm, about 21 gsm, about 25 gm, about 50 gsm, about 62 gsm, or about 71 gsm cellulose fibers. The synthetic fibers can be present in the third layer in an amount of about 1 gsm to about 50 gsm, about 5 gsm to about 35 gsm, or about 5 gsm to about 30 gsm. In particular embodiments, the first layer can include about 5 gsm, about 6 gsm, about 8 gsm, about 15 gsm, about 20 gsm, or about 27 gsm synthetic fibers.

In particular embodiments, the nonwoven material can include one or more additives such as superabsorbent polymer (SAP) disposed between, for example, the second and third layer. For example, and not by way of limitation, one or more additives can be present in an amount of about 5 gsm to about 30 gsm, about 15 gsm to about 25 gsm, or about 10 gsm to about 20 gsm. In certain embodiments, the nonwoven material can include about 10 gsm of superabsorbent polymer (SAP) disposed between the second and third layers.

Overall, the layers of the nonwoven material can have a basis weight of from about 5 gsm to about 250 gsm, or from about 30 gsm to about 200 gsm, or from about 50 gsm to about 150 gsm, or from about 50 gsm to about 65 gsm. In particular embodiments, the layers of the nonwoven material can have a basis weight of about 10 gsm, about 20 gsm, about 30 gm, about 40 gsm, about 60 gsm, about 80 gsm, about 200 gsm, or about 210 gsm.

### Features of the Topography of the Nonwoven Material

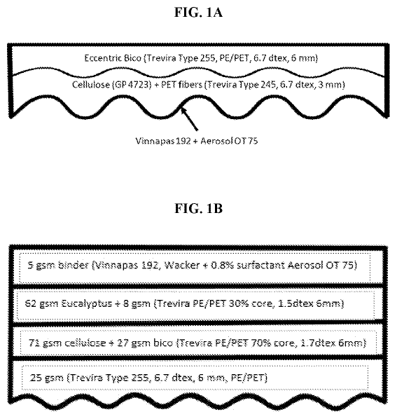

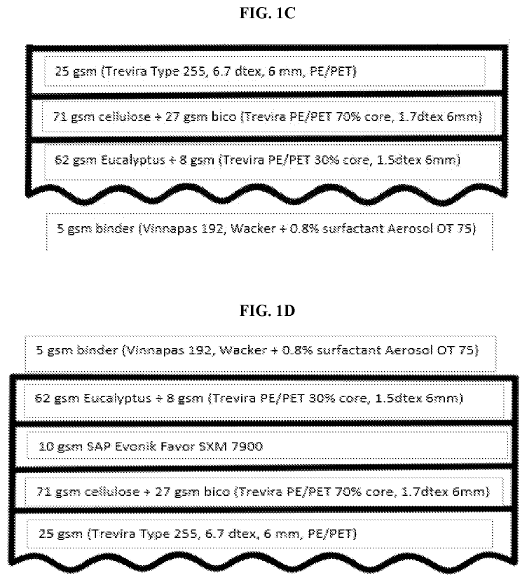

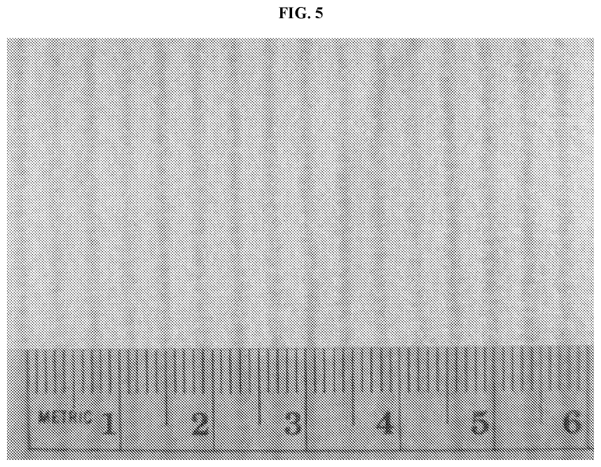

Nonwoven materials of the present disclosure can have a three-dimensional surface topography. For example, and not by way of limitation, the nonwoven material can be patterned on at least one surface. In certain embodiments, the nonwoven material can be patterned on an upper or top surface. In certain embodiments the nonwoven material can be patterned on a lower or bottom surface. In particular embodiments, the nonwoven material can be patterned on at least two surfaces. The patterning can include “ridges” and “valleys”. In certain embodiments, the ridges and valleys can be alternating. In certain embodiments, the ridges can run in the cross-machine direction (CD). In alternative embodiments, the ridges can run in the machine direction (MD). The ridges can include a high basis weight relative to the valleys of the pattern. Thus, the pattern can include areas of lower and higher basis weights of the nonwoven material, forming indentations in various shapes. For example, and not by way of limitation, the pattern can include a shape of continuous or dashed lines in various directions, dots of various dimensions, and the like. The valleys of the pattern can be about 1 mm to about 2.5 mm, about 1 mm to about 2 mm, or about 1.3 mm wide. In certain embodiments, the valleys can be at least about 2.5 mm, at least about 2 mm, at least about 1.3 mm, or at least about 1 mm wide. The ridges of the pattern can be about 2 mm to about 4 mm, about 2.1 mm to about 2.8 mm, or about 2.6 mm wide. In certain embodiments, the ridges can be at least about 4 mm, at least about 2.8 mm, at least about 2.6 mm, at least about 2.1 mm, or at least about 2 mm wide. In particular embodiments, the nonwoven material can include a three-dimensional pattern as provided in FIG. 1A. The patterning can be provided, for example, by a forming fabric having a three-dimensional topography, for example, a forming fabric including ridges running in the cross-machine direction of the forming fabric. An exemplary image of a nonwoven material formed with a patterned forming wire (e.g., Ribtech 84, Albany International, Rochester, N.H.) is provided in FIG. 5.

Nonwoven materials of the present disclosure advantageously have low runoff. Such nonwoven materials can also have adequate liquid acquisition, distribution and storage properties. The nonwoven materials of the present disclosure can include a three-dimensional pattern on at least one surface thereof. Such patterning can impart low runoff characteristics to the nonwoven material while simultaneously allowing for desired liquid acquisition, distribution and storage properties.

Nonwoven materials of the present disclosure can have low runoff. Such low runoff can be imparted onto nonwoven materials of the present disclosure through a three-dimensional pattern on at least one surface of the nonwoven material. Specifically, the presently disclosed nonwoven materials can have a low percent runoff. In certain embodiments, nonwoven materials of the present disclosure can have a percent runoff of from about 0% to about 30%, about 1% to about 30%, about 1% to about 10%, or about 1% to about 5%. In particular embodiments, nonwoven materials of the present disclosure can have a percent runoff of about 1%, about 2%, about 3%, about 4%, about 5%, about 10%, or about 15%. In certain embodiments, nonwoven materials of the present disclosure can have a percent runoff of less than about 20%, less than about 15%, less than about 10%, less than about 5%, less than about 4%, less than about 3%, less than about 2%, or less than about 1%.

In particular embodiments, the nonwoven material can include two layers. The first layer can include synthetic fibers such as eccentric bicomponent fibers (e.g., Trevira Type 255, 6.7 dtex, 6 mm, PE/PET). For example, the first layer can include from about 5 gsm to about 60 gsm, about 10 gsm to about 50 gsm, or about 26.34 gsm eccentric bicomponent fibers. The second layer can include a blend of cellulosic fibers (e.g., GP 4723, fully treated pulp from Leaf River) and synthetic fibers such as bicomponent fibers (e.g., PET, Trevira Type 245, 6.7 dtex, 3 mm). For example, the second layer can include from about 10 gsm to about 90 gsm, about 15 gsm to about 80 gsm, or about 21.36 gsm cellulosic fibers and from about 1 gsm to about 50 gsm, about 5 gsm to about 35 gsm, or about 6.28 gsm bicomponent fibers. The external surface of the second layer can be coated with a binder in the form of an emulsion (e.g., Vinnapas 192, Wacker with 0.20 gsm of surfactant Aerosol OT 75, Cytec Industries). For example, the second layer can be coated with a binder in an amount of from about 1 gsm to about 10 gsm, about 1 gsm to about 8 gsm, or about 6.02 gsm. The second layer can be patterned on at least a portion of its outer surface. For example and not by way of limitation, the total basis weight of the nonwoven material can be about 60 gsm.

In particular embodiments, the nonwoven material can include three layers. The first layer can include synthetic fibers such as eccentric bicomponent fibers (e.g., Trevira Type 255, 6.7 dtex, 6 mm, PE/PET). For example, the first layer can include from about 50 gsm to about 60 gsm, about 10 gsm to about 50 gsm, or about 25 gsm eccentric bicomponent fibers. The second layer can include a blend of cellulosic fibers (e.g., GP 4723, fully treated pulp from Leaf River) and synthetic fibers such as bicomponent fibers (e.g., Trevira PE/PET 70% core, 1.7 dtex, 6 mm). For example, the second layer can include from about 10 gsm to about 90 gsm, about 15 gsm to about 80 gsm, or about 71 gsm cellulosic fibers and from about 1 gsm to about 50 gsm, about 5 gsm to about 35 gsm, or about 27 gsm bicomponent fibers. The third layer can include a blend of cellulosic fibers (e.g., Eucalyptus pulp, Suzano, untreated) and synthetic fibers such as bicomponent fibers (e.g., Trevira PE/PET 30% core, 1.5 dtex, 6 mm). For example, the third layer can include from about 10 gsm to about 90 gsm, about 15 gsm to about 80 gsm, or about 62 gsm cellulosic fibers and from about 1 gsm to about 50 gsm, about 5 gsm to about 35 gsm, or about 8 gsm bicomponent fibers. The external surface of the third layer can be coated with a binder in the form of an emulsion (e.g., Vinnapas 192, Wacker+0.8% surfactant Aerosol OT 75, Cytec Industries). For example, the third layer can be coated with a binder in an amount of from about 1 gsm to about 10 gsm, about 1 gsm to about 8 gsm, or about 5 gsm. The first layer can be patterned on at least a portion of its outer surface. For example and not by way of limitation, the total basis weight of the nonwoven material can be about 198 gsm.

In particular embodiments, the nonwoven material can have three layers. The first layer can include synthetic fibers such as eccentric bicomponent fibers (e.g., Trevira Type 255, 6.7 dtex, 6 mm, PE/PET). For example, the first layer can include from about 50 gsm to about 60 gsm, about 10 gsm to about 50 gsm, or about 25 gsm eccentric bicomponent fibers. The second layer can include a blend of cellulosic fibers (e.g., GP 4723, fully treated pulp from Leaf River) and synthetic fibers such as bicomponent fibers (e.g., Trevira PE/PET 70% core, 1.7 dtex, 6 mm). For example, the second layer can include from about 10 gsm to about 90 gsm, about 15 gsm to about 80 gsm, or about 71 gsm cellulosic fibers and from about 1 gsm to about 50 gsm, about 5 gsm to about 35 gsm, or about 27 gsm bicomponent fibers. The third layer can include a blend of cellulosic fibers (e.g., Eucalyptus pulp, Suzano, untreated) and synthetic fibers such as bicomponent fibers (e.g., Trevira PE/PET 30% core, 1.5 dtex, 6 mm). For example, the third layer can include from about 10 gsm to about 90 gsm, about 15 gsm to about 80 gsm, or about 62 gsm cellulosic fibers and from about 1 gsm to about 50 gsm, about 5 gsm to about 35 gsm, or about 8 gsm bicomponent fibers. The external surface of the third layer can be coated with a binder in the form of an emulsion (e.g., Vinnapas 192, Wacker+0.8% surfactant Aerosol OT 75, Cytec Industries). For example, the external surface of the third layer can be coated with a binder in an amount of from about 1 gsm to about 10 gsm, about 1 gsm to about 8 gsm, or about 5 gsm. The third layer can be patterned on at least a portion of its outer surface. For example and not by way of limitation, the total basis weight of the nonwoven material can be about 198 gsm.

In particular embodiments, the nonwoven material can include three layers and a layer of one or more additives. The first layer can include synthetic fibers such as eccentric bicomponent fibers (e.g., Trevira Type 255, 6.7 dtex, 6 mm, PE/PET). For example, the first layer can include from about 5 gsm to about 60 gsm, from about 10 gsm to about 50 gsm, or about 25 gsm eccentric bicomponent fibers. The second layer can include a blend of cellulosic fibers (e.g., GP 4723, fully treated pulp from Leaf River) and synthetic fibers such as bicomponent fibers (e.g., Trevira PE/PET 70% core, 1.7 dtex, 6 mm). For example, the second layer can include from about 10 gsm to about 90 gsm, about 15 gsm to about 80 gsm, or about 71 gsm cellulosic fibers and from about 1 gsm to about 50 gsm, about 5 gsm to about 35 gsm, or about 27 gsm bicomponent fibers. A layer of one or more additives such as super absorbent polymer (SAP) (e.g., Evonik Favor SXM 7900) can be disposed between the second and third layers. For example, the layer of one or more additives can include from about 5 gsm to about 30 gsm, about 15 gsm to about 25 gsm, or about 10 gsm super absorbent polymer (SAP). The third layer can include a blend of cellulosic fibers (e.g., Eucalyptus pulp, Suzano, untreated) and synthetic fibers such as bicomponent fibers (e.g., Trevira PE/PET 30% core, 1.5 dtex, 6 mm). For example, the third layer can include from about 10 gsm to about 90 gsm, about 15 gsm to about 80 gsm, or about 62 gsm cellulosic fibers and from about 1 gsm to about 50 gsm, about 5 gsm to about 35 gsm, or about 8 gsm bicomponent fibers. The external surface of the third layer can be coated with a binder in the form of an emulsion (e.g., Vinnapas 192, Wacker+0.8% surfactant Aerosol OT 75, Cytec Industries). For example, the external surface of the third layer can be coated with binder in an amount of from about 1 gsm to about 10 gsm, about 1 gsm to about 8 gsm, or about 5 gsm. The first layer can be patterned on at least a portion of its outer surface. For example and not by way of limitation, the total basis weight of the nonwoven material can be about 208 gsm.

### Methods of Making the Nonwoven Materials

A variety of processes can be used to assemble the materials used in the practice of this disclosed subject matter to produce the materials, including but not limited to, traditional dry forming processes such as airlaying and carding or other forming technologies such as spunlace or airlace. Preferably, the materials can be prepared by airlaid processes. Airlaid processes include, but are not limited to, the use of one or more forming heads to deposit raw materials of differing compositions in selected order in the manufacturing process to produce a product with distinct strata. This allows great versatility in the variety of products which can be produced.

In one embodiment, the material is prepared as a continuous airlaid web. The airlaid web is typically prepared by disintegrating or defiberizing a cellulose pulp sheet or sheets, typically by hammermill, to provide individualized fibers. Rather than a pulp sheet of virgin fiber, the hammermills or other disintegrators can be fed with recycled airlaid edge trimmings and off-specification transitional material produced during grade changes and other airlaid production waste. Being able to thereby recycle production waste would contribute to improved economics for the overall process. The individualized fibers from whichever source, virgin or recycled, are then air conveyed to forming heads on the airlaid web-forming machine. A number of manufacturers make airlaid web forming machines suitable for use in the disclosed subject matter, including Dan-Web Forming of Aarhus, Denmark, M&J Fibretech A/S of Horsens, Denmark, Rando Machine Corporation, Macedon, N.Y. which is described in U.S. Pat. No. 3,972,092, Margasa Textile Machinery of Cerdanyola del Valles, Spain, and DOA International of Wels, Austria. While these many forming machines differ in how the fiber is opened and air-conveyed to the forming wire, they all are capable of producing the webs of the presently disclosed subject matter. The Dan-Web forming heads include rotating or agitated perforated drums, which serve to maintain fiber separation until the fibers are pulled by vacuum onto a foraminous forming conveyor or forming wire. In certain embodiments, the forming wire can be patterned, e.g., Ribtech 84 (Albany International, Rochester, N.H.). Various patterns are suitable for use with a forming wire. For example, and not by way of limitation, the forming wire can have a pattern including grooves. In particular embodiments, the forming wire can be used as a forming fabric. In the M&J machine, the forming head is basically a rotary agitator above a screen. The rotary agitator may comprise a series or cluster of rotating propellers or fan blades. Other fibers, such as a synthetic thermoplastic fiber, are opened, weighed, and mixed in a fiber dosing system such as a textile feeder supplied by Laroche S. A. of Cours-La Ville, France. In particular embodiments, such airlaid machines can be equipped with customized forming heads or heads capable of layer individualized longer fibers. From the textile feeder, the fibers are air conveyed to the forming heads of the airlaid machine where they are further mixed with the comminuted cellulose pulp fibers from the hammer mills and deposited on the continuously moving forming wire. Where defined layers are desired, separate forming heads may be used for each type of fiber. Alternatively or additionally, one or more layers can be prefabricated prior to being combined with additional layers, if any.

The airlaid web is transferred from the forming wire to a calendar or other densification stage to densify the web, if necessary, to increase its strength and control web thickness. In one embodiment, the fibers of the web are then bonded by passage through an oven set to a temperature high enough to fuse the included thermoplastic or other binder materials. In a further embodiment, secondary binding from the drying or curing of a latex spray or foam application occurs in the same oven. The oven can be a conventional through-air oven, be operated as a convection oven, or may achieve the necessary heating by infrared or even microwave irradiation. In particular embodiments, the airlaid web can be treated with additional additives before or after heat curing.

In certain embodiments, one or more plasticizers such as polyethylene glycol can be applied on a cellulose sheet before being disintegrated in hammermills or it can be applied by spraying on the airlaid web either during the forming process or at the end of the airlaid line after the curing of the binders has been completed. The use silicone-based chemicals can be sprayed onto the web either when it is being formed or at the end of the Airlaid line. Polyethylene glycol polymers are hydrophilic unlike silicone-based chemicals and can also be more economical than silicones.

### Applications and End Uses

The nonwoven materials of the disclosed subject matter can be used for any application known in the art. For example, the nonwoven materials can be used either alone or as a component in a variety of absorbent articles. In certain aspects, the nonwoven materials can be used in absorbent articles that absorb and retain body fluids. Such absorbent articles include baby diapers, adult incontinence products, sanitary napkins, feminine hygiene products, personal care products, and the like.

In other aspects, the nonwoven materials can be used alone or as a component in other consumer products. For example, the nonwoven materials can be used in absorbent cleaning products, such wipes, sheets, towels and the like.

### 6. EXAMPLES

The following examples are merely illustrative of the presently disclosed subject matter and they should not be considered as limiting the scope of the subject matter in any way.

### Example 1: Low Runoff Nonwoven Materials

The present Example provides for absorbent nonwoven materials of the present disclosure and methods of making the same. Structures 1, 3 and 6 were used as control structures. Structures 2, 4, 5 and 7 were used as experimental structures. Such nonwoven materials with pattern advantageously had reduced runoff characteristics.

Structure 1 was a two-layered nonwoven airlaid material formed using a pilot drum-forming machine and a flat forming fabric. The top layer included 26.34 gsm of eccentric bicomponent fibers (Trevira Type 255, 6.7 dtex, 6 mm, PE/PET). The bottom layer included a mixture of 6.28 gsm of PET fibers (Trevira Type 245, 6.7 dtex, 3 mm) and 21.36 gsm of cellulose fibers (GP 4723, fully treated pulp from Leaf River) which was bonded with a 6.02 gsm of polymeric binder in the form of an emulsion (Vinnapas 192, Wacker) with 0.20 gsm of surfactant (Aerosol OT 75, Cytec Industries).

The composition of Structure 1 is shown in Table 1.

TABLE 1
Structure 1 Composition

| | Type of | | Basis Weight |
|---|---|---|---|
| Layer | Material | Raw Materials | (gsm) |
| 1 | Synthetic | Eccentric bico (Trevira Type 255, | 26.34 |
| | | 6.7 dtex, 6 mm, PE/PET) | |
| 2 | Synthetic | PET (Trevira Type 245, 6.7 dtex, 3 | 6.28 |
| | | mm) | |
| | Pulp | Cellulose (GP 4723, fully treated | 21.36 |
| | | pulp from Leaf River) | |
| | Binder | Vinnapas 192, Wacker with 0.20 | 6.02 |
| | | gsm of surfactant Aerosol OT 75, | |
| | | Cytec Industries | |
| | | Total | 60.00 |

Structure 2 was a two-layered nonwoven airlaid material formed using a pilot drum-forming machine and a patterned wire as a forming fabric which included grooves. The top layer included 26.34 gsm of eccentric bicomponent fibers (Trevira Type 255, 6.7 dtex, 6 mm, PE/PET). The bottom layer included a mixture of 6.28 gsm of PET fibers (Trevira Type 245, 6.7 dtex, 3 mm) and 21.36 gsm of cellulose fibers (GP 4723, fully treated pulp from Leaf River) which was bonded with a 6.02 gsm of polymeric binder in the form of an emulsion (Vinnapas 192, Wacker) with 0.20 gsm of surfactant (Aerosol OT 75, Cytec Industries).

The composition of Structure 2 is shown in Table 2 and FIG. 1A. FIG. 1A schematically depicts grooves incorporated into the structure.

TABLE 2
Structure 2 Composition

| | Type of | | Basis Weight |
|---|---|---|---|
| Layer | Material | Raw Materials | (gsm) |
| 1 | Synthetic | Eccentric bico (Trevira Type 255, | 26.34 |
| | | 6.7 dtex, 6 mm, PE/PET) | |
| 2 | Synthetic | PET (Trevira Type 245, 6.7 dtex, 3 | 6.28 |
| | | mm) | |
| | Pulp | Cellulose (GP 4723, fully treated | 21.36 |
| | | pulp from Leaf River) | |
| | Binder | Vinnapas 192, Wacker with 0.20 | 6.02 |
| | | gsm of surfactant Aerosol OT 75, | |
| | | Cytec Industries | |
| | | Total | 60.00 |

Structure 3 was a three-layered nonwoven airlaid material in accordance with the disclosed subject matter formed using a pilot drum-forming machine and a flat forming fabric. The top layer included 25 gsm of eccentric bicomponent fibers (Trevira Type 255, 6.7 dtex, 6 mm, PE/PET). The intermediate layer included a mixture of 27 gsm of bicomponent fibers (Trevira PE/PET 70% core, 1.7 dtex, 6 mm) and 71 gsm of cellulose fibers (GP 4723, fully treated pulp from Leaf River). The bottom layer included a mixture of 62 gsm of Eucalyptus pulp (Suzano, untreated) and 8 gsm bicomponent fibers (Trevira PE/PET 30% core, 1.5 dtex, 6 mm) which was bonded with 5 gsm of a polymeric binder in the form of an emulsion (Vinnapas 192, Wacker+0.8% surfactant Aerosol OT 75, Cytec Industries).

The composition of Structure 3 is shown in Table 3.

TABLE 3
Structure 3 Composition

| | Type of | | Basis Weight |
|---|---|---|---|
| Layer | Material | Raw Materials | (gsm) |
| 1 | Synthetic | Eccentric bico (Trevira Type 255, | 25.0 |
| | | 6.7 dtex, 6 mm, PE/PET) | |
| 2 | Synthetic | Bico (Trevira PE/PET 70% core, | 27.0 |
| | | 1.7 dtex, 6 mm) | |
| | Pulp | Cellulose (GP 4723, fully treated | 71.0 |
| | | pulp from Leaf River) | |
| 3 | Synthetic | Bico (Trevira PE/PET 30% core, | 8.00 |
| | | 1.5 dtex, 6 mm) | |
| | Pulp | Eucalyptus pulp (Suzano, | 62.00 |
| | | untreated) | |
| | Binder | Vinnapas 192, Wacker + 0.8% | 5.00 |
| | | surfactant Aerosol OT 75, Cytec | |
| | | Industries | |
| | | Total | 198.00 |

Structure 4 was a three-layered nonwoven airlaid material formed using a pilot drum-forming machine and a patterned wire as a forming fabric which included grooves. Structure 4 was formed with the all synthetic layer on the patterned wire. The top layer included 25 gsm of eccentric bicomponent fibers (Trevira Type 255, 6.7 dtex, 6 mm, PE/PET). The intermediate layer included a mixture of 27 gsm of bicomponent fibers (Trevira PE/PET 70% core, 1.7 dtex 6 mm) and 71 gsm of cellulose fibers (GP 4723, fully treated pulp from Leaf River). The bottom layer included a mixture of 62 gsm of Eucalyptus pulp (Suzano, untreated) and 8 gsm bicomponent fibers (Trevira PE/PET 30% core, 1.5 dtex 6 mm) which was bonded with 5 gsm of a polymeric binder in the form of an emulsion (Vinnapas 192, Wacker+0.8% surfactant Aerosol OT 75, Cytec Industries).

The composition of Structure 4 is shown in Table 4 and FIG. 1B. FIG. 1B schematically depicts grooves incorporated into the structure.

TABLE 4
Structure 4 Composition

| | Type of | | Basis Weight |
|---|---|---|---|
| Layer | Material | Raw Materials | (gsm) |
| 1 | Synthetic | Eccentric bico (Trevira Type 255, | 25.0 |
| | | 6.7 dtex, 6 mm, PE/PET) | |
| 2 | Synthetic | Bico (Trevira PE/PET 70% core, | 27.0 |
| | | 1.7 dtex, 6 mm) | |
| | Pulp | Cellulose (GP 4723, fully treated | 71.0 |
| | | pulp from Leaf River) | |
| 3 | Synthetic | Bico (Trevira PE/PET 30% core, | 8.00 |
| | | 1.5 dtex, 6 mm) | |
| | Pulp | Eucalyptus pulp (Suzano, | 62.00 |
| | | untreated) | |
| | Binder | Vinnapas 192, Wacker + 0.8% | 5.00 |
| | | surfactant Aerosol OT 75, Cytec | |
| | | Industries | |
| | | Total | 198.00 |

Structure 5 is a three-layered nonwoven airlaid material formed using a pilot drum-forming machine and a patterned wire as a forming fabric which included grooves. This sample was formed with the Eucalyptus layer on the patterned wire. The top layer included 25 gsm of eccentric bicomponent fibers (Trevira Type 255, 6.7 dtex, 6 mm, PE/PET). The intermediate layer included a mixture of 27 gsm of bicomponent fibers (Trevira PE/PET 70% core, 1.7 dtex, 6 mm) and 71 gsm of cellulose fibers (GP 4723, fully treated pulp from Leaf River). The bottom layer included a mixture of 62 gsm of Eucalyptus pulp (Suzano, untreated) and 8 gsm bicomponent fibers (Trevira PE/PET 30% core, 1.5 dtex, 6 mm) which was bonded with 5 gsm of a polymeric binder in the form of an emulsion (Vinnapas 192, Wacker+0.8% surfactant Aerosol OT 75, Cytec Industries).

The composition of Structure 5 is shown in Table 5 and FIG. 1C. FIG. 1C schematically depicts grooves incorporated into the structure.

TABLE 5
Structure 5 Composition

| | Type of | | Basis Weight |
|---|---|---|---|
| Layer | Material | Raw Materials | (gsm) |
| 1 | Synthetic | Eccentric bico (Trevira Type 255, | 25.0 |
| | | 6.7 dtex, 6 mm, PE/PET) | |
| 2 | Synthetic | Bico (Trevira PE/PET 70% core, | 27.0 |
| | | 1.7 dtex, 6 mm) | |
| | Pulp | Cellulose (GP 4723, fully treated | 71.0 |
| | | pulp from Leaf River) | |
| 3 | Synthetic | Bico (Trevira PE/PET 30% core, | 8.00 |
| | | 1.5 dtex, 6 mm) | |
| | Pulp | Eucalyptus pulp (Suzano, | 62.00 |
| | | untreated) | |
| | Binder | Vinnapas 192, Wacker + 0.8% | 5.00 |
| | | surfactant Aerosol OT 75, Cytec | |
| | | Industries | |
| | | Total | 198.00 |

Structure 6 is a three-layered nonwoven airlaid material with super absorbent polymer (SAP) formed using a pilot drum-forming machine and a flat forming fabric. The top layer included 25 gsm of eccentric bicomponent fibers (Trevira Type 255, 6.7 dtex, 6 mm, PE/PET). The intermediate layer included a mixture of 25 gsm of bicomponent fibers (Trevira PE/PET 70% core, 1.7 dtex, 6 mm) and 65 gsm of cellulose fibers (GP 4723, fully treated pulp from Leaf River). A 10 gsm layer of super absorbent polymer (Evonik Favor SXM 7900) was added using a Christy feeder between the bottom and intermediate layers. The bottom layer included a mixture of 62 gsm of Eucalyptus pulp (Suzano, untreated) and 8 gsm bicomponent fibers (Trevira, PE/PET 30% core, 1.5 dtex, 6 mm) which was bonded with 5 gsm of a polymeric binder in the form of an emulsion (Vinnapas 192, Wacker+0.8% surfactant Aerosol OT 75, Cytec Industries).

The composition of Structure 6 is shown in Table 6.

TABLE 6
Structure 6 Composition

| | Type of | | Basis Weight |
|---|---|---|---|
| Layer | Material | Raw Materials | (gsm) |
| 1 | Synthetic | Eccentric bico (Trevira Type 255, | 25.0 |
| | | 6.7 dtex, 6 mm, PE/PET) | |
| 2 | Synthetic | Bico (Trevira PE/PET 70% core, | 27.0 |
| | | 1.7 dtex, 6 mm) | |
| | Pulp | Cellulose (GP 4723, fully treated | 71.0 |
| | | pulp from Leaf River) | |
| | SAP | Evonik Favor SXM 7900 | 10.00 |
| 3 | Synthetic | Bico (Trevira PE/PET 30% core, | 8.00 |
| | | 1.5 dtex, 6 mm) | |
| | Pulp | Eucalyptus pulp (Suzano, | 62.00 |
| | | untreated) | |
| | Binder | Vinnapas 192, Wacker + 0.8% | 5.00 |
| | | surfactant Aerosol OT 75, Cytec | |
| | | Industries | |
| | | Total | 208.00 |

Structure 7 was a three-layered nonwoven airlaid material with super absorbent polymer (SAP) formed using a pilot drum-forming machine and a patterned wire as a forming fabric which included grooves. Structure 7 was formed with the synthetic layer on the patterned wire. The top layer included 25 gsm of eccentric bicomponent fibers (Trevira Type 255, 6.7 dtex, 6 mm, PE/PET). The intermediate layer included a mixture of 25 gsm of bicomponent fibers (Trevira PE/PET 70% core, 1.7 dtex, 6 mm) and 65 gsm of cellulose fibers (GP 4723, fully treated pulp from Leaf River). A 10 gsm layer of super absorbent polymer (SAP) (Evonik Favor SXM 7900) was added using a Christy feeder between the bottom and intermediate layers. The bottom layer included a mixture of 62 gsm of Eucalyptus pulp (Suzano, untreated) and 8 gsm of bicomponent fibers (Trevira PE/PET 30% core, 1.5 dtex, 6 mm) which was bonded with 5 gsm of a polymeric binder in the form of an emulsion (Vinnapas 192, Wacker+0.8% surfactant Aerosol OT 75, Cytec Industries).

The composition of Structure 7 is provided in Table 7 and FIG. 1D. FIG. 1D schematically depicts grooves incorporated into the structure.

TABLE 7
Structure 7 Composition

| | Type of | | Basis Weight |
|---|---|---|---|
| Layer | Material | Raw Materials | (gsm) |
| 1 | Synthetic | Eccentric bico (Trevira Type 255, | 25.0 |
| | | 6.7 dtex, 6 mm, PE/PET) | |
| 2 | Synthetic | Bico (Trevira PE/PET 70% core, | 27.0 |
| | | 1.7 dtex, 6 mm) | |
| | Pulp | Cellulose (GP 4723, fully treated | 71.0 |
| | | pulp from Leaf River) | |
| | SAP | Evonik Favor SXM 7900 | 10.00 |
| 3 | Synthetic | Bico (Trevira PE/PET 30% core, | 8.00 |
| | | 1.5 dtex, 6 mm) | |
| | Pulp | Eucalyptus pulp (Suzano, | 62.00 |
| | | untreated) | |
| | Binder | Vinnapas 192, Wacker + 0.8% | 5.00 |
| | | surfactant Aerosol OT 75, Cytec | |
| | | Industries | |
| | | Total | 208.00 |

### Example 2: Runoff Testing (Structures 1-7)

The present Example provides runoff testing of the absorbent nonwoven materials of Example 1. Structures 1, 3 and 6 were used as control structures. Structures 2, 4, 5 and 7 were used as experimental structures.

Hygiene products manufacturers use a variety of test methods to determine the effectiveness of their products. One of such tests is a runoff test. The runoff test involves placing a sample of an absorbent material on a 30 degree plane and insulting the sample with 5 mL of synthetic blood at a rate of 38 mL/min. The less blood runs off the sample the more desirable its performance is and lower likelihood of leakages when the tested material is used as a component of a finished hygiene absorbent product.

Runoff Testing

The runoff was measured on an 8″×2.5″ sample for Structures 1-7. The sample was lined (samples were either compacted with 4 bars of pressure or not compacted at all prior to testing) with the bottom edge of the 30 degree angled plexiglass plate with the eccentric bicomponent fiber layer as the topside of the structure. The sample was then taped to the plate. The sample was insulted with 5 mL of synthetic blood (from Johnson, Moen and Co. Inc; Lot #=528181; received in May 2018; product identification=ASTM F1670 Synthetic Blood, having viscosity of 5.56 cPs and surface tension of 40-44 dynes/cm; chemicals=Acrysol G111: ammonium polyacrylate polymer, Twitchell 6808 surfactant, Direct Red Azo Dye 081, and HPLC distilled water) by turning on the pump for 7.9 seconds. The pump delivered the synthetic blood at a flow rate of 38 mL/min. The point of insult was at a horizontal distance of 5 cm from the bottom of the sample. The tube delivering the synthetic blood was 1 cm above the sample. Synthetic blood, that was not readily absorbed by the sample, ran off onto a plastic weigh boat and was weighed to give a runoff weight. Percent run-off was then calculated.

Structures 1 and 2

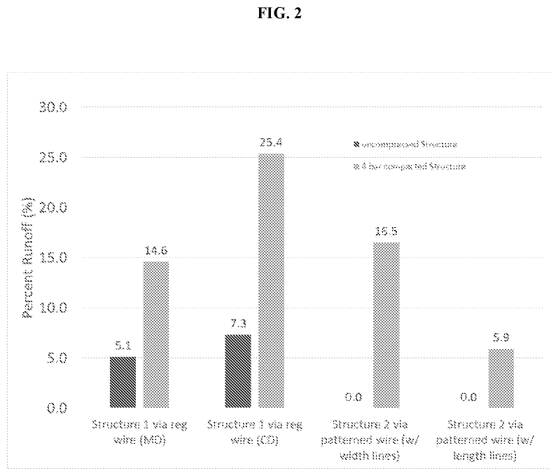

Structures 1 and 2 were tested for runoff Structure 1 was a control sample. The runoff test was performed on Structure 1 cut in the machine direction. The runoff test was then performed on pieces of Structure 1 cut in the cross direction. Likewise, the runoff test was performed on Structure 2 cut such that the lines were running along the length of the sample. These lines are denoted as “length lines” in FIG. 2. The runoff test was performed on Structure 2 cut such that the lines were running from side to side or the width of the sample. These lines are displayed as “width lines” in FIG. 2. Afterward, the four preceding tests were repeated on the samples after they have been compacted with 4 bars of pressure. The term “lines” herein is used to describe the textured pattern observed from the bottom side of Structure 2 that appeared to be visible lines. Lines created by low basis weight areas in Structure 2 represent valleys and lines created by high basis weight areas of Structure 2 represent peaks.

The test results for Samples 1 and 2 are provided in FIG. 2.

The uncompressed structures of Structure 2 (shown in FIG. 1B) are significantly more adept at preventing runoff than the uncompressed structures of Structure 1 (shown in FIG. 1A). This unexpected improvement in the runoff performance can be achieved with the textured surface being on the opposite position to the surface acquiring the liquid and is irrespective of the direction of the pattern. The role the textured surface plays in the runoff is further displayed for the 4 bar compacted structures. The 4 bar compacted Structure 1 produced with a flat wire and cut in the cross direction has significantly higher runoff than 4 bar compacted Structure 2 produced with a patterned wire and also cut in the cross direction such that the lines are running from side to side or the width of the sample.

Structures 3-5

Structures 3-5 were tested for runoff. Structure 3 was a control sample. The runoff test was performed on Structure 3 cut in the machine direction. The runoff test was then performed on pieces of Structures 4 and 5 cut in the machine and cross-machine directions. Machine direction samples are denoted as having “width lines” as the lines run the full width of the sample. Cross-machine direction samples are denoted as having “length lines” as the lines run the full length of the sample. Afterward, the five preceding tests were repeated on the samples after they have been compacted with 4 bars of pressure.

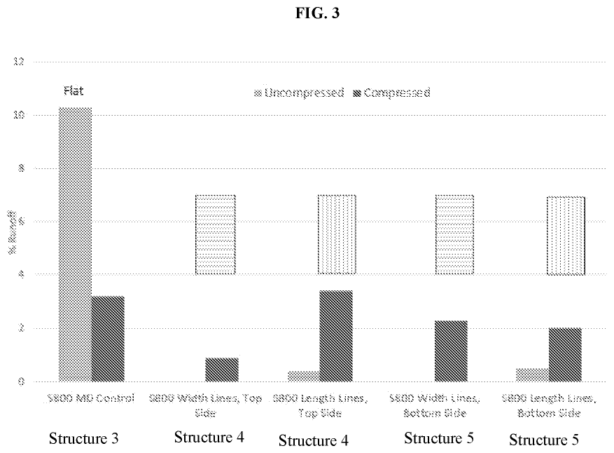

The test results for Samples 3-5 are provided in FIG. 3.

All uncompressed S800 sample runoffs with the patterned wire had increased performance as compared to the samples made on the flat forming wire. All compressed samples runoffs are the same or improved than the control. This data also shows that the improved runoff can be provided with the pattern on the top and on the bottom of the structure.

Structures 6-7

Structures 6-7 were tested for runoff. Structure 6 was a control sample. The runoff test was performed on Structure 6 cut in the machine direction. The runoff test was then performed on pieces of Structure 7 cut in the machine and cross-machine directions. Machine direction samples are denoted as having “width lines” as the lines run the full width of the sample. Cross-machine direction samples are denoted as having “length lines” as the lines run the full length of the sample. Afterward, the three preceding tests were repeated on the samples after they have been compacted with 4 bars of pressure.

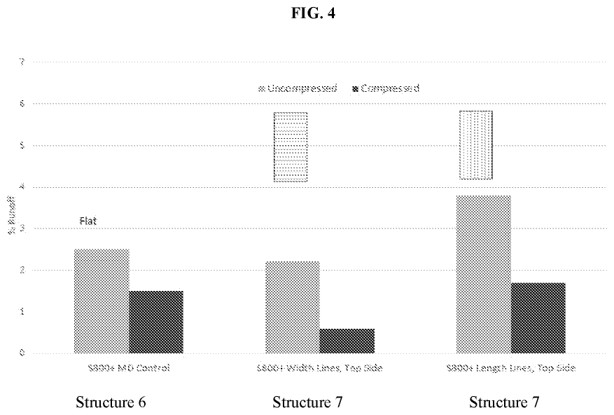

The test results for Samples 6-7 are provided in FIG. 4.

Both Structure 7 samples, compressed and uncompressed with width lines, had increased performance as compared the control Structure 6 as far as runoff. Both Structure 7 samples, compressed and uncompressed with length lines performed less than the Structure 6 control samples. A shown in FIG. 4, once super absorbent polymer (SAP) is added to the structure, all the compressed samples exhibited increased performance as compared to the uncompressed samples.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

1. An airlaid nonwoven material comprising:
a first layer comprising bicomponent fibers, and
a second layer disposed adjacent to the first layer, the second layer comprising cellulose fibers and bicomponent fibers,
wherein the second layer is bonded on at least a portion of its outer surface with a binder, wherein at least a portion of the second layer is patterned, and
wherein the nonwoven material has a percent runoff of less than about 5%.

2. The airlaid nonwoven material of claim 1, wherein the patterning of the second layer comprises alternating ridges and valleys, and the ridges have a higher basis weight than the valleys.

3. The airlaid nonwoven material of claim 2, wherein the ridges are from about 2 mm to about 4 mm wide and the valleys are from about 1 mm to about 2.5 mm wide.

4. An absorbent article comprising the airlaid nonwoven material of claim 1.

5. An airlaid nonwoven material comprising:
a first layer comprising bicomponent fibers,
a second layer disposed adjacent to the first layer, the second layer comprising cellulose fibers and bicomponent fibers, and
a third layer disposed adjacent to the second layer, the third layer comprising cellulose fibers and bicomponent fibers,
wherein the third layer is bonded on at least a portion of its outer surface with a binder, wherein at least a portion of at least one of the first and third layers is patterned, and wherein the nonwoven material has a percent runoff of less than about 5%.

6. The airlaid nonwoven material of claim 5, wherein the patterning of at least one of the first and third layers comprises alternating ridges and valleys, and the ridges have a higher basis weight than the valleys.

7. The airlaid nonwoven material of claim 6, wherein the ridges are from about 2 mm to about 4 mm wide and the valleys are from about 1 mm to about 2.5 mm wide.

8. An absorbent article comprising the airlaid nonwoven material of claim 5.

9. An airlaid nonwoven material comprising:
a first layer comprising bicomponent fibers,
a second layer disposed adjacent to the first layer, the second layer comprising cellulose fibers and bicomponent fibers,
a third layer disposed adjacent to the second layer, the third layer comprising eucalyptus fibers and bicomponent fibers, and
a layer of super absorbent polymer disposed between the second and third layers, wherein the third layer is bonded on at least a portion of its outer surface with a binder, wherein at least a portion of the first layer is patterned, and
wherein the nonwoven material has a percent runoff of less than about 5%.

10. The airlaid nonwoven material of claim 9, wherein the patterning of the first layer comprises alternating ridges and valleys, and the ridges have a higher basis weight than the valleys.

11. The airlaid nonwoven material of claim 10, wherein the ridges are from about 2 mm to about 4 mm wide and the valleys are from about 1 mm to about 2.5 mm wide.

12. An absorbent article comprising the airlaid nonwoven material of claim 9.

13. An airlaid nonwoven material comprising:
a first layer comprising synthetic fibers, and
a second layer disposed adjacent to the first layer, the second layer comprising cellulose

14. The airlaid nonwoven material of claim 13, wherein the nonwoven material has a percent runoff of less than about 1%.

15. The airlaid nonwoven material of claim 13, further comprising a third layer disposed adjacent to the second layer, the third layer comprising cellulose fibers and synthetic fibers.

16. The airlaid nonwoven material of claim 15, further comprising a layer of superabsorbent polymer disposed between the second and third layers.

17. The airlaid nonwoven material of claim 13, wherein the second layer is bonded on at least a portion of its outer surface with a binder.

18. The airlaid nonwoven material of claim 15, wherein the third layer is bonded on at least a portion of its outer surface with a binder.

19. The airlaid nonwoven material of claim 15, wherein the cellulose fibers of the third layer comprise eucalyptus fibers.

20. The airlaid nonwoven material of claim 13, wherein the synthetic fibers of the first and second layers comprise bicomponent fibers.

21. An absorbent article comprising the airlaid nonwoven material of claim 13.

22. An airlaid nonwoven
material comprising: a first layer
comprising synthetic fibers,
a second layer disposed adjacent to the first layer, the second layer comprising cellulose fibers and synthetic fibers, and
a third layer disposed adjacent to the second layer, the third layer comprising cellulose fibers and synthetic fibers,
wherein the nonwoven material is patterned on at least a portion of at least one surface,
and
wherein the nonwoven material has a percent runoff of less than about 5%.

23. The airlaid material of claim 22, wherein the nonwoven material has a percent runoff of less than about 1%.

24. The nonwoven material of claim 22, further comprising a layer of superabsorbent polymer disposed between the second and third layers.

25. The nonwoven material of claim 22, wherein the third layer is bonded on at least a portion of its outer surface with a binder.

26. The nonwoven material of claim 22, wherein the cellulose fibers of the third layer comprise eucalyptus fibers.

27. An absorbent article comprising the nonwoven material of claim 22.
